(19) United States
(12) Patent Application Publication
(10) Pub. No.: US 2013/0274555 A1
(43) Pub. Date: Oct. 17, 2013

(54) MEMBER FOR MEDICAL DEVICE

(71) Applicant: Olympus Corporation, Tokyo (JP)

(72) Inventors: Hirokazu Kamioka, Tokyo (JP); Takeshi Iizuka, Tokyo (JP)

(21) Appl. No.: 13/913,936

(22) Filed: Jun. 10, 2013

Related U.S. Application Data
(63) Continuation of application No. PCT/JP2012/059188, filed on Apr. 4, 2012.

(30) Foreign Application Priority Data
Apr. 6, 2011 (JP) 2011-084644

(51) Int. Cl. A61B 1/005

(52) U.S. Cl. CPC A61B 1/005
USPC 600/139

(57) ABSTRACT

An object of the present invention is to provide a member for a medical device that is imparted with flexibility and has superior damage tolerance even when reduced in thickness. The present invention provides a member for a medical device molded from a material composed of multiple components, wherein at least one of the multiple components is an elastomer, and at least one of the multiple components has rebound resilience of 1% to 30%.

### TECHNICAL FIELD

The present invention relates to a member for a medical device.

The present application claims priority on the basis of Japanese Patent Application No. 2011-084644 filed in Japan on Apr. 6, 2011, the contents of which are incorporated herein by reference.

The present application is a U.S. continuing patent application based on the PCT International Patent Application, PCT/JP2012/59188, filed on Apr. 4, 2012, the contents of which are incorporated herein by reference.

### RELATED ART

Members for medical devices such as the curved portions of endoscopes and catheters frequently undergo coating treatment on the outer shell thereof to prevent malfunctions caused by leakage of water such as when inserted into the body or during cleaning.

For example, a soft elastomer material such as a fluorine-based elastomer or silicone-based elastomer is used for the outer shell of the curved portions of endoscopes described in Japanese Unexamined Patent Application, First Publication No. 2010-104668.

### ABSTRACT OF THE INVENTION

As a result of conducting extensive studies, the inventors of the present invention found that by using a material composed of multiple components that contains an elastomer as at least one component thereof and further contains at least one component having rebound resilience of 1% to 30%, a member for a medical device can be obtained that is able to absorb external impacts, and as a result thereof, is imparted with flexibility and is resistant to tearing and the formation of holes even if reduced in thickness, thereby leading to completion of the present invention.

Namely, the member for a medical device according to one embodiment of the present invention is a member for a medical device formed from a material composed of multiple components, comprising an elastomer, as at least one of the multiple components, and a component, as at least one of the multiple components, having rebound resilience of 1% to 30%.

In addition, the overall rebound resilience of the aforementioned material composed of multiple components is preferably 1% to 30%.

Moreover, the aforementioned at least one of the multiple components having rebound resilience of 1% to 30% is preferably a polymer compound.

In addition, the aforementioned material composed of multiple components preferably contains two or more types of elastomers having different rebound resilience, and the aforementioned at least one of the multiple components having rebound resilience of 1% to 30% is preferably an elastomer.

Moreover, the member for a medical device of the present invention is preferably molded into the shape of a tube or sheet.

### PREFERRED EMBODIMENTS OF THE INVENTION

The following provides a detailed explanation of an embodiment of the present invention.

The member for a medical device according to one embodiment of the present invention is formed from a material composed of multiple components, comprising an elastomer, as at least one of the components, and a component, as at least one of the components, having rebound resilience of 1% to 30%.

Furthermore, in the description of the present application, rebound resilience refers to a value measured in compliance with ISO 4662.

(Elastomer Component)

At least one of the components that compose the material of the member for a medical device is an elastomer.

Since elastomers fulfill the role of impacting flexibility to a member for a medical device, a member for a medical device having superior flexibility is obtained by making at least one of the components in the material an elastomer.

Examples of elastomers include rubber (thermosetting elastomers) and thermoplastic elastomers.

Examples of rubber include natural rubber, isoprene rubber, butadiene rubber, 1,2-polybutadiene rubber, styrene-butadiene rubber, chloroprene rubber, nitrile rubber, butyl rubber, ethylene-propylene rubber, chlorosulfonated polyethylene, acrylic rubber, epichlorhydrin rubber, silicone rubber, fluorine rubber and urethane rubber.

Examples of thermoplastic elastomers include urethane-based, styrene-based, ester-based, vinyl chloride-based, olefin-based, nitrile-based and polyamide-based elastomers.

One type of these elastomers may be used alone or two or more types may be used in combination.

In the embodiment of the present invention, a thermoplastic elastomer is preferable for the elastomer from the viewpoint of improving molding workability when molding the member for a medical device.

An elastomer synthesized according to a known synthesis method or a commercially available elastomer may be used for the elastomer in the embodiment of the present invention. The following indicates examples of commercially available elastomers.

Examples of commercially available urethane-based elastomers include “ElastollanC60A” manufactured by BASF Corp.

Examples of commercially available styrene-based elastomers include “Septon Compound JL40NFS”, “Septon Compound FY35N-01” and “Septon Compound JH50N” manufactured by Kuraray Plastics Co., Ltd.

Examples of commercially available ester-based elastomers include “Hytrel SB654” and “ETPV 60A01L” manufactured by Du Pont-Toray Co., Ltd.

Furthermore, there are no particular limitations on the rebound resilience of the elastomer in the embodiment of the present invention. An elastomer having rebound resilience of less than 1% may be used, an elastomer having rebound resilience of 1% to 30% may be used, or an elastomer having rebound resilience of greater than 30% may be used.

The proportion of elastomer in the materials composed of other components in the embodiment of the present invention is preferably 5% by weight to 100% by weight and more preferably 10% by weight to 90% by weight based on 100% by weight of the material of the member for a medical device. If the proportion of elastomer is within the aforementioned ranges, a member for a medical device having adequate flexibility is easily obtained.

(Component having Rebound Resilience of 1% to 30%)

Among the components that compose the material of the member for a medical device of the embodiment of the present invention, the rebound resilience of at least one of the components is 1% to 30%.

Since this component having rebound resilience of 1% to 30% fulfills the role of imparting performance for absorbing external impacts to the member for a medical device, the member for a medical device is able to demonstrate superior damage tolerance even if the overall form of the member for a medical device is reduced in thickness.

Here, if rebound resilience is less than 1%, the material loses its rubber elasticity and has difficulty in stretching. On the other hand, if rebound resilience exceeds 30%, it becomes difficult to absorb external impacts, thereby resulting in increased susceptibility to a decrease in damage tolerance, and making this undesirable.

A polymer compound is an example of a component having rebound resilience of 1% to 30% as described above.

Examples of polymer compounds include urethane-based, styrene-based and ester-based resin compounds and the various types of elastomers previously exemplified in the explanation of the elastomer component.

One type of these polymer compounds may be used alone or two or more types may be used in combination.

Furthermore, in the case of using a resin compound for the component having rebound resilience of 1% to 30%, the resin compound may be of the same type as the aforementioned elastomer component or may be of a different type from the aforementioned elastomer component (such as the combination of a urethane-based resin compound and an ester-based elastomer component). A combination in which the resin compound and the elastomer component are of the same type is preferable from the viewpoint of compatibility.

In addition, in the case of using an elastomer for the component having rebound resilience of 1% to 30%, two or more types of elastomers having different rebound resilience can be used. In this case, there are no particular limitations on the rebound resilience of the remaining elastomer(s) provided the rebound resilience of at least one of the elastomers is within the range of 1% to 30%, and may be within the range of 1% to 30% or outside this range.

A polymer compound synthesized according to a known synthesis method or a commercially available polymer compound maybe used for the polymer compound having rebound resilience of 1% to 30%. The following indicates examples of commercially available polymer compounds having rebound resilience of 1% to 30%.

Examples of commercially available urethane-based polymer compounds include the urethane-based resin “Elastollan NY90A” manufactured by BASF Corp.

Examples of commercially available styrene-based polymer compounds include the styrene-based elastomer “Septon Compound JL40NFS” manufactured by Kuraray Plastics Co., Ltd.

Examples of commercially available ester-based polymer compounds include the ester-based elastomer “Hytrel SB654” manufactured by Du Pont-Toray Co., Ltd.

The proportion of the compound having rebound resilience of 1% to 30% to the entire material composed of multiple components in the embodiment of the present invention is preferably 5% by weight to 100% by weight and more preferably 10% by weight to 90% by weight based on 100% by weight of the material of the member for a medical device. If the proportion of the compound having rebound resilience of 1% to 30% is within the aforementioned ranges, a member for a medical device that is able to adequately absorb external impacts is easily obtained.

(Carbon)

The material of the member for a medical device may also contain a colorant in the form of carbon in addition to the aforementioned elastomer component and component having rebound resilience of 1% to 30%. In addition to fulfilling the role of a colorant, carbon also fulfills the role of a reinforcing agent. The containing of carbon not only allows the obtaining of coloring effects, but makes it easy to adjust the member for a medical device to a desired hardness or improve heat resistance of the member for a medical device according to the content thereof.

The proportion of carbon is preferably 0.5% by weight to 10% by weight based on 100% by weight of the material of the member for a medical device. If the proportion of carbon is 0.5% by weight or more, coloring effects are adequately obtained. On the other hand, if the proportion of carbon is 10% by weight or less, the member for a medical device can be inhibited from becoming excessively hard.

(Silica)

The material of the member for a medical device may further contain silica. Since silica fulfills the role of a reinforcing agent, the containing of silica has the same effect as in the case of containing the aforementioned carbon, and more specifically, allows the obtaining of effects that make it easy to adjust the member for a medical device to a desired hardness or improve heat resistance of the member for a medical device.

The proportion of silica is preferably 0.05% by weight to 50% by weight and more preferably 0.5% by weight to 15% by weight based on 100% by weight of the material of the member for a medical device. If the proportion of silica is 0.05% by weight or more, reinforcing effects are adequately obtained. On the other hand, if the proportion of silica is 50% by weight or less, the member for a medical device can be inhibited from becoming excessively hard.

(Other Components)

Various types of arbitrary components such as fillers or fibers in the form of optional components may also be contained in the material of the member for a medical device of the embodiment of the present invention.

Examples of fillers include inorganic fillers such as barium sulfate, titanium oxide, aluminum oxide, calcium carbonate, calcium silicate, magnesium silicate or aluminum silicate; and organic fillers such as polytetrafluoroethylene resin, polyethylene resin, polypropylene resin, phenol resin, polyimide resin, melamine resin or silicone resin.

One type of these fillers may be used alone or two or more types may be used in combination.

Examples of fibers include inorganic fibers such as asbestos, glass fiber, alumina fiber or rock wool; and organic fibers such as cotton, wool, silk, hemp, nylon fiber, aramid fiber, vinylon fiber, polyester fiber, rayon fiber, acetate fiber, phenol-formaldehyde fiber, polyphenylene sulfide fiber, acrylic fiber, polyvinyl chloride fiber, polyvinylidene chloride fiber, polyurethane fiber or tetrafluoroethylene fiber.

One type of these fibers may be used alone or two or more types may be used in combination.

(Production Method)

The member for a medical device of the embodiment of the present invention can be produced using various commonly used methods.

First, the elastomer component and the component having rebound resilience of 1% to 30% are compounded with a twin-screw roller, kneader or Banbury mixer and the like followed by the addition of carbon, silica and arbitrary components as necessary while mixing therein to prepare a material of a member for a medical device.

The overall rebound resilience of the material obtained according to the aforementioned process is preferably 1% to 30%. If the rebound resilience of the material is within the range of 1% to 30%, the rebound resilience of a member for a medical device molded from this material has the same value as the rebound resilience of the material (namely, 1% to 30%), thereby allowing a member for a medical device having superior balance between flexibility and damage tolerance to be easily obtained.

Rebound resilience of the material can be adjusted by adjusting the types and incorporated amounts of each component that composes the material.

Next, the resulting material is used to mold to a desired shape. Known rubber molding methods such as injection molding or extrusion molding can be used for the molding method. For example, the material can be filled into a metal mold of a desired shape followed by hot pressing and cooling.

There are no particular limitations on the shape of the member for a medical device, and the shape can be suitably selected corresponding to the particular application, examples of which include a tube, sheet, rod, ring and various types of blocks.

Since the member for a medical device of the embodiment of the present invention as explained above is molded from a material composed of multiple components as previously described, it is able to absorb external impacts. Accordingly, it is imparted with flexibility, has superior damage tolerance, and is resistant to tearing and the formation of holes even if reduced in thickness.

Furthermore, the material of the member for a medical device may also be composed of two or more types of elastomers having different rebound resilience. In this case, the rebound resilience of at least one of the elastomers is 1% to 30%.

Although the member for a medical device of the embodiment of the present invention is suitable for use as a member of an endoscope or catheter, for example, it is particularly preferably used for the outer shell of an endoscope, a bending prevention member of an endoscope, a switch button or outer shell covering a switch button of an endoscope, and an O-ring used inside an endoscope.

### EXAMPLES

Although the following provides a more detailed explanation of the present invention through examples thereof, the present invention is not limited thereto.

Raw materials and evaluation methods used in the examples and comparative examples are as indicated below.

[Raw Materials]

The types, trade names, manufacturers and rebound resilience values of each component (raw material) that composes materials of members for a medical device are shown in Table 1.

TABLE 1

| | | | Rebound |
|---|---|---|---|
| Type | Trade Name | Manufacturer | Resilience (%) |
| Urethane-based | Elastollan C60A | BASF | 50 |
| elastomer | | | |
| Urethane-based | Elastollan NY90A | BASF | 26 |
| resin | | | |
| Styrene-based | Septon Compound | Kuraray Plastics | 8 |
| elastomer 1 | JL40NFS | | |
| Styrene-based | Septon Compound | Kuraray Plastics | 55 |
| elastomer 2 | FY35N-01 | | |
| Styrene-based | Septon Compound | Kuraray Plastics | 56 |
| elastomer 3 | JH50N | | |
| Ester-based | Hytrel SB654 | Du Pont-Toray | 27 |
| elastomer 1 | | | |
| Ester-based | ETPV 60A01L | Du Pont-Toray | 48 |
| elastomer 2 | | | |
| Fluorine-based | Dai-El DC40-70 | Daikin | 60 |
| elastomer | | Industries, Ltd. | |
| Silicone-based | KE-961-U/C-8 | Du Pont-Toray | 55 |
| elastomer | (vulcanizing | | |
| | agent) | | |
| Silica | Min-U-Sil #5 | U.S. Silica Co. | — |

[Measurement/Evaluation]

<Measurement of Rebound Resilience>

Rebound resilience was measured according to a rebound resilience test in compliance with ISO 4662.

<Measurement of Tensile Strength>

Tensile strength was measured according to a tensile test in compliance with JIS K 6251.

<Measurement of Tear Strength>

Tear strength was measured according to a tear strength test in compliance with JIS K 6252.

<Measurement of Hardness>

Durometer hardness was measured in compliance with JIS K 6252.

<Evaluation of Damage Tolerance>

A cracking test was carried out by allowing a pin having a diameter of 1.5 mm attached to a 70 g weight to drop vertically from a height of 110 mm onto a molded product formed into the shape of a tube having a thickness of 0.5 mm, followed by visually observing the formation of cracks after testing and evaluating cracking using the evaluation criteria indicated below.

◯: No occurrence of cracking

×: Occurrence or cracking

### Example 1

Each component was placed in a twin screw kneader equipped with screws having a screw diameter of 20 mm in accordance with the blending composition shown in Table 2 followed by melting and kneading under conditions of a temperature of 200° C. to prepare a pelletized material. Next, the resulting pelletized material was molded into the shape of a sheet having a thickness of 2 mm using a single-screw extrusion molding machine to obtain a molded product. The resulting molded product was measured for rebound resilience, tensile strength, tear strength and hardness. The results are shown in Table 2.

### Examples 2 to 7 and Comparative Examples 1 to 4

Materials were prepared in the same manner as Example 1 with the exception of changing the blending composition of each component to the compositions shown in Table 2, followed by producing molded products in the form of sheets and tubes, and carrying out each measurement and evaluation in the same manner as Example 1. The results are shown in Table 2.

TABLE 2

| Blending Composition (parts by weight) | | | | | | | | | | | | | |
|---|---|---|---|---|---|---|---|---|---|---|---|---|---|
| | | Rebound | | | | | | | | | | | |
| | | resilience | Examples | | | | | | | Comparative Examples | | | |
| | Type | (%) | 1 | 2 | 3 | 4 | 5 | 6 | 7 | 1 | 2 | 3 | 4 |
| Materials | Urethane-based | 50 | 80 | 0 | 80 | 0 | 0 | 0 | 0 | 0 | 0 | 0 | 0 |
| | elastomer | | | | | | | | | | | | |
| | Urethane-based | 26 | 20 | 0 | 20 | 0 | 0 | 0 | 80 | 0 | 0 | 0 | 0 |
| | resin | | | | | | | | | | | | |
| | Styrene-based | 8 | 0 | 60 | 0 | 80 | 20 | 0 | 0 | 0 | 0 | 100 | 0 |
| | elastomer 1 | | | | | | | | | | | | |
| | Styrene-based | 55 | 0 | 40 | 0 | 20 | 80 | 0 | 0 | 0 | 0 | 0 | 20 |
| | elastomer 2 | | | | | | | | | | | | |
| | Styrene-based | 56 | 0 | 0 | 0 | 0 | 0 | 0 | 0 | 0 | 0 | 0 | 80 |
| | elastomer 3 | | | | | | | | | | | | |
| | Ester-based | 27 | 0 | 0 | 0 | 0 | 0 | 90 | 20 | 0 | 0 | 0 | 0 |
| | elastomer 1 | | | | | | | | | | | | |
| | Ester-based | 48 | 0 | 0 | 0 | 0 | 0 | 10 | 0 | 0 | 0 | 0 | 0 |
| | elastomer 2 | | | | | | | | | | | | |
| | Fluorine-based | 60 | 0 | 0 | 0 | 0 | 0 | 0 | 0 | 100 | 0 | 0 | 0 |
| | elastomer | | | | | | | | | | | | |
| | Silicone-based | 55 | 0 | 0 | 0 | 0 | 0 | 0 | 0 | 0 | 100 | 0 | 0 |
| | elastomer | | | | | | | | | | | | |
| | Silica | — | 0 | 0 | 10 | 0 | 0 | 0 | 0 | 0 | 0 | 0 | 0 |

| | | | | | | | | | | | |
|---|---|---|---|---|---|---|---|---|---|---|---|
| Rebound resilience (%) | 28 | 25 | 28 | 13 | 30 | 29 | 26 | 60 | 55 | 8 | 56 |
| Tensile strength (MPa) | 15 | 13 | 17 | 13 | 18 | 9 | 14 | 17 | 7 | 8.1 | 9 |
| Tear strength (kN/m) | 45 | 35 | 47 | 25 | 27 | 30 | 37 | 28 | 18 | 30 | 32 |
| Hardness (Shore) | 64A | 59A | 67A | 52A | 73A | 63A | 75A | 68A | 65A | 40A | 60A |
| Damage tolerance (cracking test) | ◯ | ◯ | ◯ | ◯ | ◯ | ◯ | ◯ | X | X | X | X |

As is clear from Table 2, the molded products obtained in each of the examples did not demonstrate the occurrence of cracking in the cracking test and had superior damage tolerance.

On the other hand, the molded products obtained in each of the comparative examples demonstrated the occurrence of cracking in the cracking test and had inferior damage tolerance.

1. A member for a medical device formed from a material composed of multiple components, comprising:
an elastomer, as at least one of the multiple components; and
a component, having rebound resilience of 1% to 30%, as at least one of the multiple components .

2. The member for a medical device according to claim 1, having an entire shape of a tube.

3. The member for a medical device according to claim 1, wherein the overall rebound resilience of the material composed of multiple components is 1% to 30%.

4. The member for a medical device according to claim 1, wherein the component having rebound resilience of 1% to 30%, as at least one of the multiple components is a polymer compound.

5. The member for a medical device according to claim 1, wherein the material composed of multiple components contains two or more types of elastomers having different rebound resilience, and
the component having rebound resilience of 1% to 30%, as at least one of the multiple components, is an elastomer.

6. The member for a medical device according to claim 1, wherein rebound resilience is a value measured in compliance with ISO 4662.
